(19) United States
(12) Patent Application Publication
(10) Pub. No.: US 2026/0013786 A1
(43) Pub. Date: Jan. 15, 2026

(54) ULTRA THIN SMART ELECTROPALATOGRAPHY SYSTEM TO ASSIST LINGUISTIC AND MEDICAL DIAGNOSIS

(71) Applicant: University of Florida Research Foundation, Inc., Gainesville, FL (US)

(72) Inventors: Yong Kyu Yoon, Gainesville, FL (US); Ariel David Cerpa, Gainesville, FL (US); Ziqi Jia, Gainesville, FL (US); Saeyeong Jeon, Gainesville, FL (US); Sunghyun Hwang, Gainesville, FL (US)

(21) Appl. No.: 19/238,626

(22) Filed: Jun. 16, 2025

Related U.S. Application Data
(60) Provisional application No. 63/669,269, filed on Jul. 10, 2024.

(51) Int. Cl. A61B 5/00; G16H 50/70

(52) U.S. Cl. CPC A61B 5/4803; A61B 5/682; G16H 50/70

(57) ABSTRACT

In one aspect, the disclosure relates to a pseudo-palate for capacitive sensing of tongue proximity to, contact with, and pressure against a subject's hard palate, the pseudo-palate comprising an upper and a lower plurality of tracing lines; wherein the upper and lower pluralities of tracing lines are embedded in a flexible material and separated by gap comprising the flexible material; wherein the upper and the lower pluralities of tracing lines are connected by vias located at a plurality of intersection points; and wherein, when the pseudo-palate is worn by the subject, the upper plurality of tracing lines is closer to the hard palate than is the lower plurality of tracing lines. The pseudo-palate is under 30 μm thick to reduce interference of the pseudo-palate with the subject's speech and communicates wirelessly with a computer to monitor data in real time. Also disclosed are methods of making and using the pseudo-palate.

### CROSS-REFERENCE TO RELATED APPLICATIONS

This application claims the benefit of U.S. Provisional Application Ser. No. 63/669,269, filed Jul. 10, 2024, which is incorporated herein by reference in its entirety.

### STATEMENT REGARDING FEDERALLY SPONSORED RESEARCH OR DEVELOPMENT

This invention was made with government support under grant number 2037266 awarded by the National Science Foundation. The government has certain rights in the invention.

### BACKGROUND

Electropalatography (EPG) is a system that monitors contact patterns between the tongue and hard palate during articulation. It has wide applications in articulation disorders and other types of disease involving speaking difficulties. Therapists have used an EPG system to help patients with speech sound disorder (SSD) improve quality of life; to improve recovery and correct residual articulation disorder for cleft palate patient after surgery; to identify and improve the unclear articulation pattern for Down syndrome patients; to improve the pronunciation of hearing disorder speakers; and to evaluate recovery and accelerate rehabilitation for patients suffering from traumatic brain injury.

Besides its traditional applications, an EPG system, when combined with an acoustic analysis system, can capture changes in tongue movement patterns, such as reduction in the range of motion and velocity, along with other potential biomarkers for Parkinson's Disease (PD) and schizophrenia. This data can provide supporting evidence for the early diagnosis of affected individuals. Moreover, the EPG system also contributes to the advancement of human machine interface (HMI). Andreasen used an EPG system to interact with a personal computer with high accuracy, while Horne used a certain palate region on the EPG device for specific computer commands. All research indicates that EPG has high potential to be used as part of the HMI and even Virtual Reality environments.

A pseudo-palate sensor is the main component in the EPG system that has an electrode array for tongue movement detection as shown in FIG. 2A. The recorded pattern can serve as a biomarker for a range of therapy and diagnosis purpose. Some EPG devices use resistive sensors capable of detecting contact locations, while others provide pressure values. Traditional and commercial EPG devices employ metal electrodes to detect the contact between the tongue and upper palate, most of which use the resistive sensing mechanism. For example, Articulate Instruments used the resin infused electrode arrays as pseudo palate, while Smart Palate system used the polyimide packaged flexible printed circuit board.

Though the EPG device has a long history and has been extensively used, there remain challenges that prevent the broader adoption of this device. First, most commercial EPG devices are thick (2˜3 mm) and constructed with rigid materials whose bulkiness and lack of flexibility results in poor wearing comfort, which can impede natural pronunciation. While EPG devices have demonstrated three sensing modes—proximity distance sensing, contact sensing and pressure sensing—no more than two sensing methods are typically able to be integrated into a single device. The data captured by the sensor can therefore lack thoroughness and precision in charting the tongue's movement. Furthermore, the sensing resolution of an EPG device is often constrained by the channel count, typically ranging from 30 to 90, which may not provide sufficient resolution to capture detailed nuances and subtleties. Moreover, most EPG devices are tethered with wires that need to come out of mouths for data transmission and powering, limiting the patients' tongue positions and lip movement. Last, certain commercial EPG systems require customization and manual assembly, resulting in costs that can soar to several thousand dollars, making them not very economical.

Despite advances in electropalatography research, there are still no EPG devices that are thin enough to avoid impeding natural pronunciation, that can integrate all three currently known sensing methods, that offer high resolution, that do not require wires extending from the wearer's mouth, and that are inexpensive to produce. These needs and other needs are satisfied by the present disclosure.

### SUMMARY

In accordance with the purpose(s) of the present disclosure, as embodied and broadly described herein, the disclosure, in one aspect, relates to a pseudo-palate for capacitive sensing of tongue proximity to, tongue contact with, and tongue pressure against a hard palate of a subject, the pseudo-palate comprising an upper plurality of tracing lines and a lower plurality of tracing lines; wherein the upper plurality of tracing lines and the lower plurality of tracing lines are embedded in a flexible material and separated by gap comprising the flexible material; wherein the upper plurality of tracing lines and the lower plurality of tracing lines are connected by vias located at a plurality of intersection points; and wherein, when the pseudo-palate is worn by the subject, the upper plurality of tracing lines is closer to the hard palate than is the lower plurality of tracing lines. The disclosed pseudo-palate is under 30 μm thick in order to reduce interference of the pseudo-palate with the subject's natural speech patterns. In another aspect, the pseudo-palate communicates wirelessly with a computer so data from the pseudo-palate can be collected and monitored in real time. Also disclosed are methods of making the disclosed pseudo-palate and methods of using the pseudo-palate to diagnose neurological and/or speech disorders.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic demonstrating data collection and analysis of a smart electropalatography for linguistic and medical application (SELMA) device.

FIG. 2A shows a commercially available EPG device. FIG. 2B shows an fPCB version of a disclosed SELMA device and the customized read-out circuit.

FIG. 3A shows the mechanism of proximity sensing mode and FIG. 3B shows the mechanism of the contact pressure sensing modes.

FIG. 4A shows the pattern of a SELMA sensing array and FIG. 4B shows a 2 layer metal approach for the pattern in FIG. 4A.

FIG. 5A shows an exemplary proximity sensing test setup and FIG. 5B shows an exemplary contact and pressure sensing mode test setup.

FIG. 6A shows sensor response for proximity sensing. FIG. 6B shows sensor response for pressure sensing.

FIG. 7A shows a sensing pattern wirelessly processed and displayed on user interface for proximity sensing, while FIG. 7B shows the same for pressure sensing.

FIG. 8 shows the fabrication steps for a parylene process for a SELMA device.

FIGS. 9A-9B show fabrication results for a parylene packaged sensing test structure. Details under an optical microscope (FIG. 9A) and device release from the substrate (FIG. 9B).

FIG. 9C shows a full-sized parylene sensor.

FIGS. 10A-10D show COMSOL simulations and readout for an exemplary SELMA device. FIG. 10A shows a perspective view of a model tongue approaching a device having numbered sensors. FIG. 10B shows several stages of the approach of the tongue. FIG. 10C shows a side view of the model tongue approaching the device. FIG. 10D shows sample readout for change in capacitance for differently numbered terminals (see FIG. 10A) based on proximity to the approaching tongue.

FIGS. 11A-11E show several views of the electronic components of an exemplary SELMA device. FIG. 11A is a top view of a circuit board useful for communicating with a parylene sensor. FIG. 11B is a bottom view of the circuit board of FIG. 11A. FIG. 11C shows the circuit board attached by an exemplary means to a capacitive sensor array, allowing for continuous reading of capacitive sensing of tongue proximity, tongue contact, and tongue pressure on the sensor array. FIG. 11D shows a view of the circuit board demonstrating reading of sensor data and transmitting the data wirelessly through a mini-antenna (Bluetooth low energy or BLE). FIG. 11E shows an exemplary computer equipped with BLE communication function receiving data from the SELMA device and displaying the same on a screen.

FIGS. 12A-12B show a parylene thin film multimodal thin film sensor connected to a wireless board with battery from (FIG. 12A) front side and (FIG. 12B) back side.

FIG. 13 shows sensor capacitance over time for an exemplary parylene thin film sensor.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

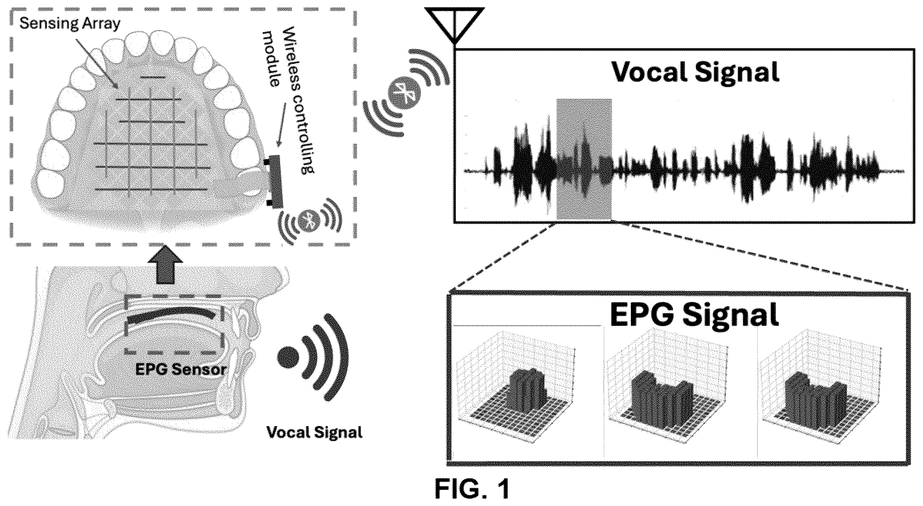

Disclosed herein is an ultra thin smart electropalatography (EPG) system. In one aspect, it can have a thickness of less than 30 μm, approximately one hundredth the thickness of a commercial device. In a further aspect, the system incorporates 112 channels and employs various sensing modes such as proximity, touching, and pressing. In an aspect, a capacitance sensing mechanism is used for all sensing modes at different sensing ranges. Exemplary features of the system include an extremely thin and flexible film sensor structure, enhancing wearer comfort; capacitive sensors capable of proximity, contact, and press sensing, indicating the relative positions of the tongue concerning the membrane; a higher channel count enabling high-resolution tongue touching maps during speech; and complete wireless supporting circuits for real-time data visualization. Also disclosed herein is a fully wireless and stand-alone read-out board (FIG. 1).

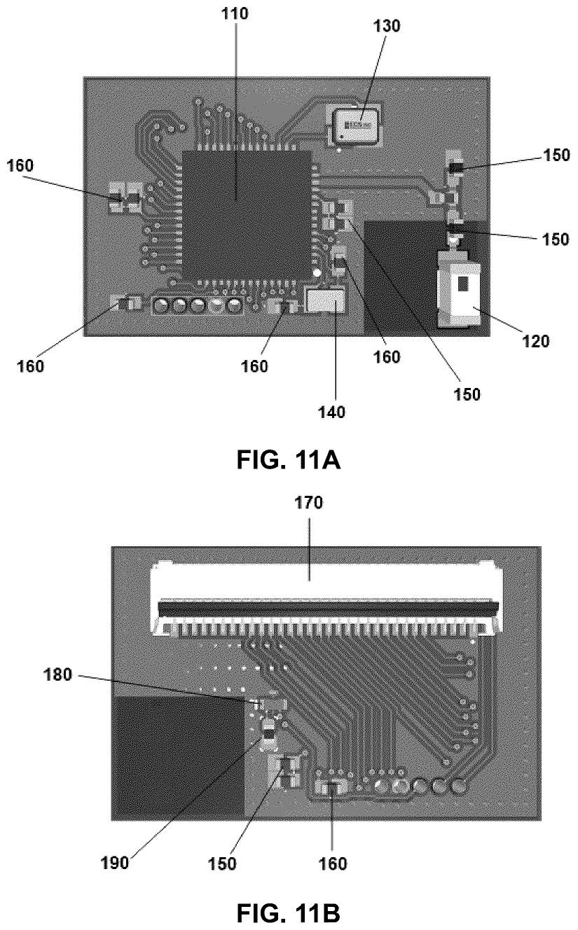

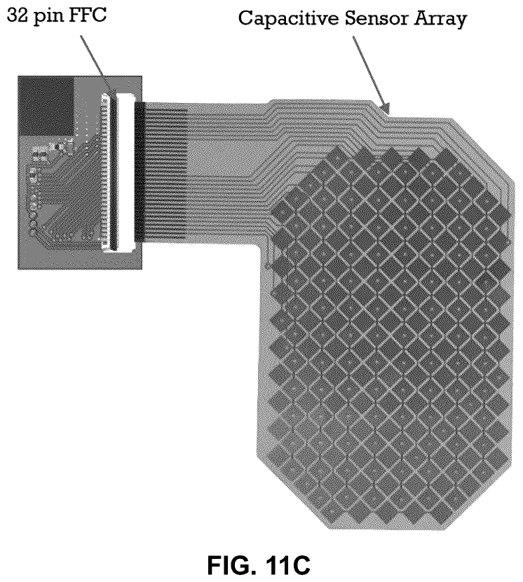

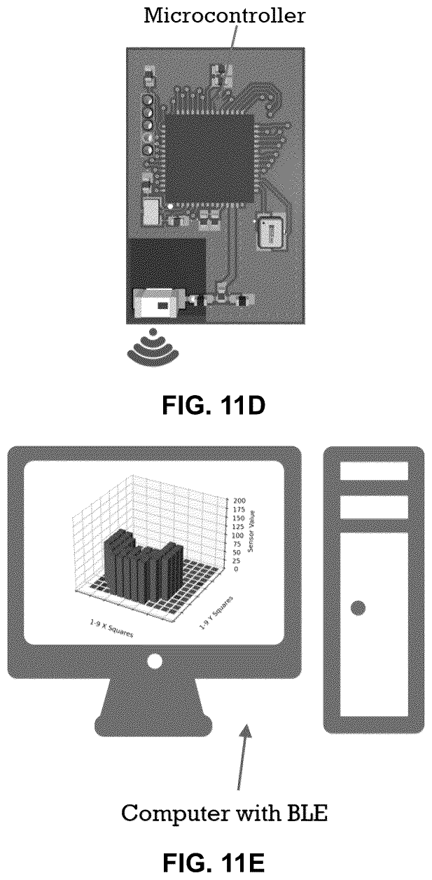

Turning to FIGS. 11A-11B, the wireless read-out board is equipped with a CYBC4247LQI-BL483 microcontroller 110 and communicates with an external computer through 2.4 GHz mini-antenna 120. The read-out board is further equipped with 32.768 KHz crystal oscillator 130, 24 MH crystal oscillator 140, inductors 150, capacitors 160, and 43 pin FFC connector 170. The read-out board is further equipped with red LED 180 and resistor 190 although other circuitry arrangements are envisioned and should also be considered disclosed. Turning to FIGS. 11C-11E, a pseudo-palate with capacitive sensor array connects to a microcontroller using a 32 pin flexible flat cable (FFC). This enables continuous reading of capacitive sensing including proximity, contact, and pressure sensing. The microcontroller reads sensor data and sends data using Bluetooth Low Energy (BLE) technology to a computer configured to receive the data and display it as a three-dimensional plot on screen.

### Pseudo-Palate

In one aspect, disclosed herein is a pseudo-palate for capacitive sensing of tongue proximity to, tongue contact with, and tongue pressure against the hard palate of a subject, the pseudo palate including an upper plurality of tracing lines and a lower plurality of tracing lines, wherein the upper plurality of tracing lines and the lower plurality of tracing lines are embedded in a flexible material and separated by gap comprising the flexible material; wherein the upper plurality of tracing lines and the lower plurality of tracing lines are connected by vias located at a plurality of intersection points; and wherein, when the pseudo-palate is worn by the subject, the upper plurality of tracing lines is closer to the hard palate than is the lower plurality of tracing lines. In a further aspect, the capacitive sensing can be fringing field capacitance sensing.

In one aspect, the flexible material can be parylene. In another aspect, the pseudo palate has a thickness of less than about 30, 29, 28, 27, 26, 25, 24, 23, 22, 21, 20, 19, 18, or 17 μm, or can be about 16 μm. In a further aspect, the pseudo-palate is considerably thinner than other EPG devices known in the art. Without wishing to be bound by theory, a thinner pseudo-palate can lead to more accurate results in EPG studies and testing since having less material against the hard palate leads to more natural speech patterns for subjects wearing the pseudo-palate.

In one aspect, the upper plurality of tracing lines are perpendicular to the lower plurality of tracing lines. In one aspect, the pseudo-palate includes at least 112 vias. In still another aspect, the tracing lines can be made from gold, titanium, platinum, nickel, chromium, copper, silver, or any combination thereof such as, for example, a titanium-gold (Ti—Au) alloy. In one aspect, the tracing lines can have a width of from about 5 μm to about 50 μm, or from about 10 μm to about 40 μm, or from about 20 μm to about 30 μm, or about 5, 10, 15, 20, 25, 30, 35, 40, 45, or about 50 μm, or a combination of any of the foregoing values, or a range encompassing any of the foregoing values. In one aspect, the tracing lines are used in place of bulky wires to avoid interference with the subject's speech patterns.

In still another aspect, the pseudo-palate further includes a microprocessor, a wireless communication module, and a battery, wherein the microprocessor, wireless communication module, and battery are positioned on an outside wall of the subject's upper gums. In still another aspect, the pseudo-palate can further include a temperature sensor, an inertia sensor, an accelerometer, a gyroscope, a magnetometer, an infrared sensor for monitoring impacts, a heart rate monitor, or a combination thereof. In one aspect, the pseudo-palate can be disposable; that is, rather than sterilizing the same device for use in multiple patients, it can be discarded after use, reducing cleaning time and removing the chance of cross-contamination between patients.

### Method for Making the Pseudo-Palate

Also disclosed herein is a method for making the disclosed pseudo-palate. In an aspect, the method includes at least the following steps:
(a) depositing a first layer of flexible material on a substrate;
(b) depositing a first metal on the first layer of flexible material;
(c) depositing a second layer of flexible material on the substrate;
(d) performing etching to create a plurality of via holes;
(e) depositing a second metal on the second layer of flexible material;
(f) depositing a third layer of flexible material on the substrate to produce the pseudo-palate;
(g) performing etching to create a plurality of openings; and
(h) releasing the pseudo-palate from the substrate.

In a further aspect, the first, second, and/or third layers of flexible material can be or include parylene. In another aspect, the substrate can be a silicon wafer. In still another aspect, the first metal, the second metal, or both can be gold, titanium, platinum, nickel, chromium, copper, silver, or any combination thereof, such as, for example, a titanium-gold (Ti—Au) alloy.

In one aspect, the method can include performing an etching step after step (b), step (f), or both step (b) and step (f), wherein the etching step removes at least a portion of the metal. In one aspect, removing at least a portion of the metal leaves a plurality of tracing lines of metal embedded in the flexible material. In still another aspect, etching in step (b) leaves a first plurality of parallel tracing lines and etching in step (f) leaves a second plurality of parallel tracing lines, wherein the first plurality of parallel tracing lines are perpendicular in orientation to the second plurality of parallel tracing lines. In one aspect, depositing a second metal on the substrate in step (e) fills the plurality of via holes with the second metal to produce a plurality of metal connections. In one aspect, the plurality of metal connections link the first plurality of parallel tracing lines with the second plurality of parallel tracing lines at a plurality of crossing points between the first plurality of parallel tracing lines and the second plurality of parallel tracing lines.

In another aspect, the plurality of openings created in step (h) are configured to receive input connections, output connections, or both. In still another aspect, etching in step (d), in step (h), or both step (d) and step (h) can be reactive ion etching.

### Method for Diagnosing a Neurological Condition

In one aspect, provided herein is a method for diagnosing a neurological condition in a subject, the method including at least the steps of:
(a) fitting the subject with the pseudo-palate of any of claims 1-13;
(b) requiring the subject to perform at least one vocal task;
(c) collecting acoustic data and data from the pseudo-palate generated by the performance of the at least one vocal task;
(d) comparing the acoustic data and data from the pseudo-palate to a database of data from healthy subjects and subjects having diagnosed neurological conditions performing the same at least one vocal task; and
(e) diagnosing the neurological condition.

In a further aspect, when the subject's tongue contacts the pseudo-palate and serves as an additional ground for the pseudo-palate, a higher electric field is found in the portion of the pseudo-palate contacted by the tongue. In another aspect, a decrease in distance between the subject's tongue and the pseudo-palate changes capacitance between the upper plurality of tracing lines and the lower plurality of tracing lines. In still another aspect, tongue contact with the pseudo-palate compresses the flexible material, reducing the gap between the upper plurality of tracing lines and the lower plurality of tracing lines.

In one aspect, the neurological condition can be a traumatic brain injury, Parkinson's disease, dyspraxia, dysarthria, congenital sensory neuropathy, schizophrenia, depression, fatigue, stress, or a combination thereof. In a further aspect, the traumatic brain injury can be a concussion. In one aspect, the vocal task can be a single task or a dual task. In a further aspect, the vocal task can be picture description, storytelling, or syllable repetition. In one aspect, the dual task can include an exercise task such as, for example, walking on a treadmill or pedaling a stationary bicycle.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a capacitive sensor,” “a sensing mode,” or “a readout pattern,” include, but are not limited to, series, combinations, or collections of two or more such capacitive sensors, sensing modes, or readout patterns, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “traumatic brain injury” refers to a blow or jolt to the head (i.e., from a sports injury, auto accident, fall, or the like) that causes a disruption in the normal functioning of the brain. Traumatic brain injury (or TBI) can also be caused by a penetrating head injury. In one aspect, a TBI may fall on a spectrum anywhere from a concussion to severe and lasting brain damage. In one aspect, the pseudo-palates disclosed herein are useful for detecting speech patterns associated with TBI prior to the onset of other symptoms.

A “concussion” is one type of TBI. Concussion symptoms can include, but are not limited to, memory problems, problems with balance and coordination, trouble concentrating, headaches, and the like. In one aspect, an early symptom of concussion is altered speech patterns. In a further aspect, the pseudo-palates disclosed herein are useful for detecting these altered speech patterns and thus for early diagnosis of concussions and other TBIs.

A “biomarker” as used herein refers to a measurable parameter indicative of a disease, injury, infection, or the like. Biomarkers can include substances in the blood (i.e., particular proteins) or can include speech, including alterations in speech patterns. In one aspect, the pseudo-palates useful herein can measure changes in speech (e.g., tongue position and pressure) that are useful as biomarkers for early detection of TBI.

“Articulatory information” as used herein refers to location, size, pressure, and duration of tongue contacts with the hard palate. In one aspect, the pseudo-palates disclosed herein can be used to collect articulatory information. In a further aspect, articulatory information can be used in a complementary fashion with acoustic information for early detection of TBIs and other neurological events.

“Electropalatography” or “EPG” refers to a technique useful for monitoring contact between the tongue and hard palate during speech. In EPG, a “pseudo-palate” is fitted against the wearer's palate. Electrodes in the pseudo-palate record location of tongue contact with palate, size of contact, pressure of the contact, and length of contact. In one aspect, the pseudo-palate disclosed herein is configured to transmit signals from the electrodes to a processing unit that can communicate with an external computer, tablet, or smartphone for analysis of the signals. Thus, in another aspect, the pseudo-palate disclosed herein and its associated accessories are highly portable and can be used, for example, on a sports field in case of suspected injury as well as in a laboratory or medical setting.

In another aspect, as used herein, “capacitive sensing” refers to an approach wherein two electrode layers are separated by a material (e.g., a flexible material) and a capacitance change induced by the change in the gap between the top and bottom electrodes or conditions proximate to the electrode layers can be measured as the tongue contacts various parts of the pseudo-palate during speech. In a further aspect, capacitance at each pixel in the sensing array can be measured and sent to a microprocessor to be assessed to determine position, size, and duration of tongue contact, as well as pressure of tongue contact with the pseudo-palate disclosed herein. In some aspects, the tongue can form a ground or contact for capacitive sensing approaches and/or can replace one of the layers of electrodes.

A “flexible material” as used herein refers to a compressible, flexible polymer or polymer system useful for fabrication of the pseudo-palates disclosed herein. In one aspect, the flexible material can be parylene, or any material capable of containing or being fabricated to contain an array of sensors for a capacitive sensing approach. In another aspect, the flexible material is comfortable for the user to wear and does not interfere with speech patterns. In still another aspect, the flexible material does not easily rip or tear during speech even if very thin. In yet another aspect, the flexible material is biocompatible.

“Parasitic capacitance” or “stray capacitance” as used herein refers to unavoidable capacitance between parts of an electronic circuit due to proximity of the parts. In some aspects, proximity of the tongue to the disclosed EPG device results in parasitic capacitance. In another aspect, this parasitic capacitance can be monitored and used in device readout for additional information regarding speech patterns.

“Place of articulation” refers, in one aspect, to the location in the mouth of tongue contact wherein the vocal tract is obstructed in order to produce a sound, or to another obstruction (such as, for example, bilabial articulation wherein the lips come together). Various regions of articulation include the epiglottis, pharynx, uvula, soft palate, hard palate, and the like. In one aspect, the pseudo-palates disclosed herein are useful for monitoring palatal articulation.

The “speech motor control system” is made up of those systems and organs responsible for the production of speech including, but not limited to, respiration, phonation, and articulation. The respiratory system as well as the larynx, pharynx, tongue, lips, mandible, and soft palate, among other systems and organs, are involved with speech motor control, as well as the basal ganglia and frontal lobes of the brain, which are involved in fine muscle control. In one aspect, some brain injuries as well as neurological disorders such as, for example, Parkinson's disease, can negatively affect the speech motor control system and, thus, the quality of speech produced. In a further aspect, the pseudo-palate disclosed herein can be used to assess and identify changes in speech and speech motor control at an early stage in cases of suspected brain injury or neurological disorders, potentially leading to earlier and more successful medical interventions.

“Wireless” as used herein refers to a lack of wired connections between devices such as, e.g., a lack of wires connecting the disclosed pseudo-palate to any device, computer, monitoring equipment, or the like outside the body of the subject wearing the pseudo-palate. Wireless communications can be carried out over wireless links such as, but not limited to, RF, Wi-Fi, Bluetooth®, cellular, or other appropriate wireless technologies. The wireless pseudo-palate disclosed herein can incorporate a microprocessor, wireless communication module, battery, and/or other sensors within the pseudo-palate and include necessary electrical connections to power those devices and read data from them.

As used herein, an “embedded” component is surrounded on all sides by the material in which it is embedded. For example, if a metal layer, component, or printed element is embedded in a polymeric material, the metal layer, component, or printed element is completely surrounded by the polymeric material. In some aspects, a small portion (e.g., less than 10% or less than 5%) of the embedded component can be externally exposed in order to make an electrical connection for input, output, power supply, or the like, but the majority of the embedded component is still surrounded by the polymeric material.

As used herein, an individual's “hard palate” refers to that portion of the anatomy of the mouth that separates the oral cavity from the nasal cavity. Put another way, the hard palate forms the roof of the oral cavity and the floor of the nasal cavity. In one aspect, the hard palate is a thin, horizontal, bony plate. In another aspect, the teeth do not form a part of the hard palate.

As used herein, “tracing lines” or “traces” refers to highly conductive tracks used to connect components on a printed circuit board. In one aspect, tracing lines allow electrical charge to flow through the circuit board. In a further aspect, tracing lines or traces function in place of wires and have the advantage over wires of being much thinner, thereby reducing the overall thickness of the disclosed device. In still another aspect, use of tracing lines or traces instead of wires reduces device interference in speech patterns, thereby rendering the results obtained from the disclosed pseudo-palate more accurate and more useful in diagnosis of conditions related to speech pathologies.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A pseudo-palate for capacitive sensing of tongue proximity to, tongue contact with, and tongue pressure against a hard palate of a subject, the pseudo-palate comprising an upper plurality of tracing lines and a lower plurality of tracing lines;
wherein the upper plurality of tracing lines and the lower plurality of tracing lines are embedded in a flexible material and separated by gap comprising the flexible material;
wherein the upper plurality of tracing lines and the lower plurality of tracing lines are connected by vias located at a plurality of intersection points, wherein the vias are filled with metal; and
wherein, when the pseudo-palate is worn by the subject, the upper plurality of tracing lines is closer to the hard palate than is the lower plurality of tracing lines.

Aspect 2. The pseudo-palate of aspect 1, wherein the capacitive sensing comprises fringing field capacitance sensing.

Aspect 3. The pseudo-palate of aspect 1 or 2, wherein the flexible material comprises parylene.

Aspect 4. The pseudo-palate of any one of aspects 1-3, wherein the pseudo-palate has a thickness of less than about 30 μm.

Aspect 5. The pseudo-palate of any one of aspects 1-4, wherein the pseudo-palate has a thickness of about 16 μm.

Aspect 6. The pseudo-palate of any one of aspects 1-5, wherein the upper plurality of tracing lines are perpendicular to the lower plurality of tracing lines.

Aspect 7. The pseudo-palate of any one of aspects 1-6, wherein the pseudo-palate comprises at least 112 vias.

Aspect 8. The pseudo-palate of aspect any one of aspects 1-7, wherein the tracing lines comprise gold, titanium, platinum, nickel, chromium, copper, silver, or a combination thereof.

Aspect 9. The pseudo-palate of aspect 8, wherein the tracing lines comprise a titanium-gold (Ti—Au) alloy.

Aspect 10. The pseudo-palate of any one of aspects 1-9, wherein the tracing lines comprise a width of from about 5 μm to about 50 μm.

Aspect 11. The pseudo-palate of any one of aspects 1-10, further comprising a microprocessor, a wireless communication module, and a battery, wherein the microprocessor, wireless communication module, and battery are positioned on an outside wall of the subject's upper gums.

Aspect 12. The pseudo palate of any one of aspects 1-11, further comprising a temperature sensor, an inertia sensor, an accelerometer, a gyroscope, a magnetometer, an infrared sensor for monitoring impacts, a heart rate monitor, or a combination thereof.

Aspect 13. The pseudo palate of any one of aspects 1-12, wherein the pseudo-palate is disposable.

Aspect 14. A method for making a pseudo-palate, the method comprising:
(a) depositing a first layer of flexible material on a substrate;
(b) depositing a first metal on the first layer of flexible material;
(c) depositing a second layer of flexible material on the substrate;
(d) performing etching to create a plurality of via holes;
(e) depositing a second metal on the second layer of flexible material;
(f) depositing a third layer of flexible material on the substrate to produce the pseudo-palate;
(g) performing etching to create a plurality of openings; and
(h) releasing the pseudo-palate from the substrate.

Aspect 15. The method of aspect 14, wherein the first layer of flexible material, second layer of flexible material, third layer of flexible material, or any combination thereof comprises parylene.

Aspect 16. The method of aspect 14 or 15, wherein the substrate comprises a silicon wafer.

Aspect 17. The method of any one of aspects 14-16, wherein the first metal, the second metal, or both comprise gold, titanium, platinum, nickel, chromium, copper, silver, or any combination thereof.

Aspect 18. The method of any one of aspects 14-17, wherein the first metal, the second metal, or both comprise a titanium-gold (Ti—Au) alloy.

Aspect 19. The method of aspect any one of aspects 14-18, further comprising performing an etching step after step (b), step (f), or both step (b) and step (f), wherein the etching step removes at least a portion of the metal.

Aspect 20. The method of any one of aspects 14-19, wherein removing at least a portion of the metal leaves a plurality of parallel tracing lines of metal embedded in the flexible material.

Aspect 21. The method of any one of aspects 14-20, wherein etching in step (b) leaves a first plurality of parallel tracing lines and etching in step (f) leaves a second plurality of parallel tracing lines, wherein the first plurality of parallel tracing lines are perpendicular in orientation to the second plurality of parallel tracing lines.

Aspect 22. The method of any one of aspects 14-21, wherein depositing a second metal on the substrate in step (e) fills the plurality of via holes with the second metal to produce a plurality of metal connections.

Aspect 23. The method of aspect 22, wherein the plurality of metal connections link the first plurality of parallel tracing lines with the second plurality of parallel tracing lines at a plurality of crossing points between the first plurality of parallel tracing lines and the second plurality of parallel tracing lines.

Aspect 24. The method of any one of aspects 14-23, wherein the plurality of openings created in step (h) are configured to receive input connections, output connections, or both.

Aspect 25. The method of any one of aspects 14-24, wherein etching in step (d), in step (h), or both step (d) and step (h) comprises reactive ion etching.

Aspect 26. A method for diagnosing a neurological condition in a subject, the method comprising:
(a) fitting the subject with the pseudo-palate of any of aspects 1-13;
(b) requiring the subject to perform at least one vocal task;
(c) collecting acoustic data and data from the pseudo-palate generated by the performance of the at least one vocal task;
(d) comparing the acoustic data and data from the pseudo-palate to a database of data from healthy subjects and subjects having diagnosed neurological conditions performing the same at least one vocal task; and
(e) diagnosing the neurological condition.

Aspect 27. The method of aspect 26, wherein the subject's tongue contacts the pseudo-palate and serves as an additional ground for the pseudo-palate, resulting in a higher electric field in a portion of the pseudo-palate contacted by the tongue.

Aspect 28. The method of aspect 26 or 27, wherein a decrease in distance between the subject's tongue and the pseudo-palate changes capacitance between the upper plurality of tracing lines and the lower plurality of tracing lines.

Aspect 29. The method of any one of aspects 26-28, wherein tongue contact with the pseudo-palate compresses the flexible material, reducing the gap between the upper plurality of tracing lines and the lower plurality of tracing lines.

Aspect 30. The method of any one of aspects 26-29, wherein the neurological condition comprises a traumatic brain injury, Parkinson's disease, dyspraxia, dysarthria, congenital sensory neuropathy, schizophrenia, depression, fatigue, stress, or a combination thereof.

Aspect 31. The method of aspect 30, wherein the traumatic brain injury comprises a concussion.

Aspect 32. The method of any one of aspects 26-31, wherein the vocal task comprises a single task.

Aspect 33. The method of any one of aspects 26-31, wherein the vocal task comprises a dual task.

Aspect 34. The method of aspect 32 or 33, wherein the vocal task comprises picture description, story-telling, or syllable repetition.

Aspect 35. The method of aspect 33 or 34, wherein the dual task comprises an exercise task.

Aspect 36. The method of aspect 35, wherein the exercise task comprises walking on a treadmill or pedaling a stationary bicycle.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: SELMA System Design

### Sensing Mechanism

The capacitance sensing mechanism stems from the electromagnetic field interactions which can be described by the Maxwell equations. Capacitive as discussed herein focuses on electric field behavior in the low-frequency regime leading to a quasi-static approximation of the equations. The simplified equation of Gauss's Law for electricity is

$$∇ \cdot E = \frac{ρ}{ϵ} \qquad ( 1 )$$

In this equation, E, ρ, and ϵ represent the electric field, the charge density, and the permittivity of the medium, respectively. This equation describes how the electric field diverges from charges. Also, the electric field can be derived from the gradient of a scalar potential ϕ,

$$E = - ∇ ϕ \qquad ( 2 )$$

By combining these principles, the Poisson Equation for electrostatics is shown as

$$- ∇ \cdot ( ϵ ∇ ϕ ) = ρ \qquad ( 3 )$$

This equation relates the electric potential in a certain area to the distribution of electric charge.

In practice, capacitive sensors measure changes in the electric field or potential by detecting the displacement current, which can be linked to the charge on an electrode with the surface area Γ as

$$q = {\int }_{Γ} ϵ E dr \qquad ( 4 )$$

Therefore, the capacitance C can be determined using the charge q and the voltage V through the relationship C=q/V.

Capacitive sensors utilize two primary methodologies for sensing: mutual capacitance and self-capacitance. In mutual capacitance sensing, an electrical signal is applied to one electrode, designated as the transmitter, while a separate electrode acts as the receiver. Objects introduced into the space between these two electrodes disrupt the electric field, modifying the resultant displacement currents and, consequently, the measured capacitance values.

Conversely, in self-capacitance sensing, a single electrode serves as both the transmitter and receiver simultaneously. This configuration assesses the electric charge that the electrode can sustain relative to a ground reference. The proximity of an object alters this capacitance by providing an alternative electric field path, thereby effectively increasing the effective capacitive “plate” area between the electrode and ground. Essentially, the object assumes a role similar to that of the second electrode in mutual capacitance sensing, affecting the displacement current in a similar manner.

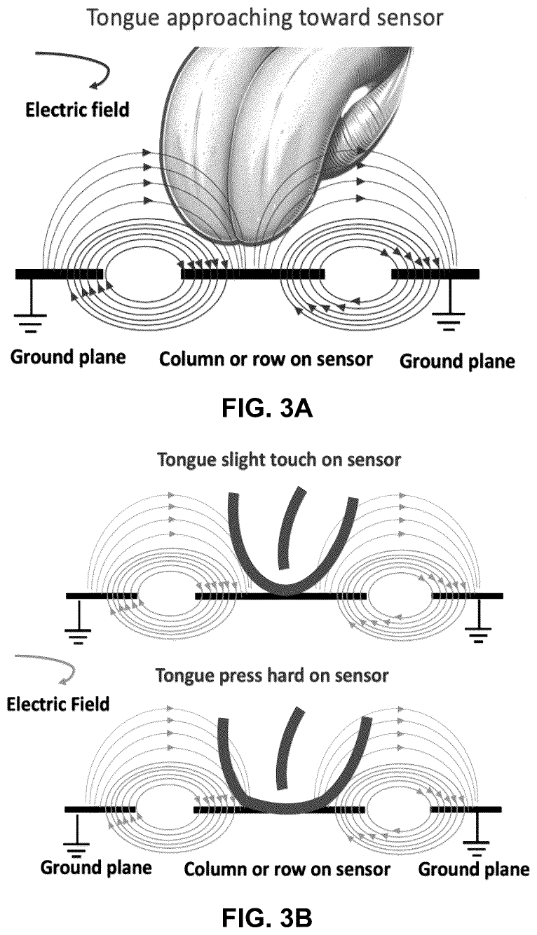

The SELMA sensor employs the self-capacitance sensing approach and would have proximity and pressure sensing by using the fringing field capacitance as shown in FIG. 3A, (b). The sensor will experience a higher electric field when the tongue acts as the ground, potentially interfering with the existing electric field between the sensor ground and sensor pad.

Upon touching in FIG. 3B, the tongue's deformation increases coverage area, and the sensor's top layer becomes thinner, enabling the detection of contact and pressure.

### SELMA Device Design

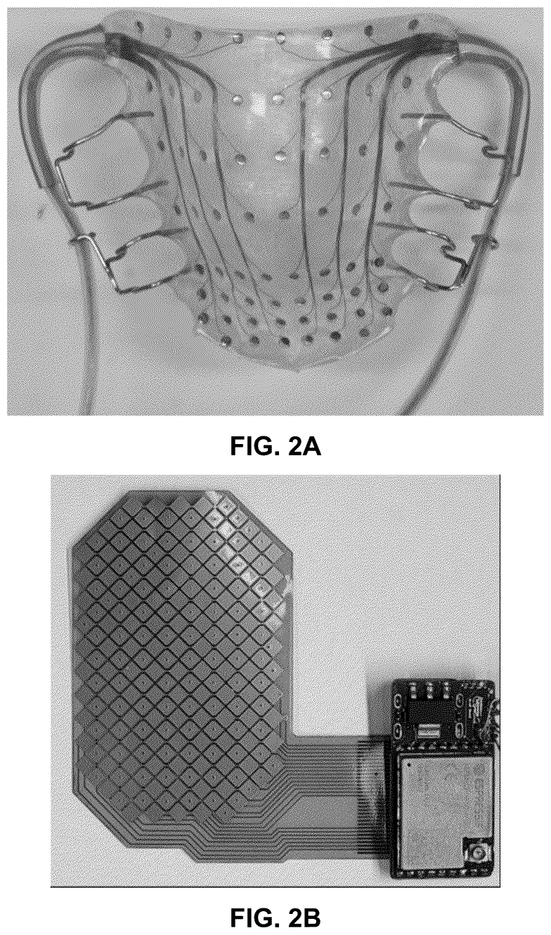

The new EPG system concept is shown in FIG. 1, where the thin film sensor array detects capacitive signal changes. The signals are transferred to the local microcontroller for data processing, further wirelessly transmitted through Bluetooth to a PC or smartphone for visualization. Previously, a flexible EPG device with active shielding layer made from silver nanowire (AgNW) and Polydimethylsiloxane (PDMS) was reported. Herein, a new capacitive sensing array pattern with 112 channels on flexible printed circuit board (fPCB) with a customized readout circuit for untethered wireless operation as shown in FIG. 2B was tested.

A crossed array pattern shows two ground planes serving as the sensing structure, where each column and row acts as an independent capacitor with ground. As shown in FIG. 3A, when the tongue approaches and touches the sensor, its proximity changes the parasitic capacitance between the metal pad and ground.

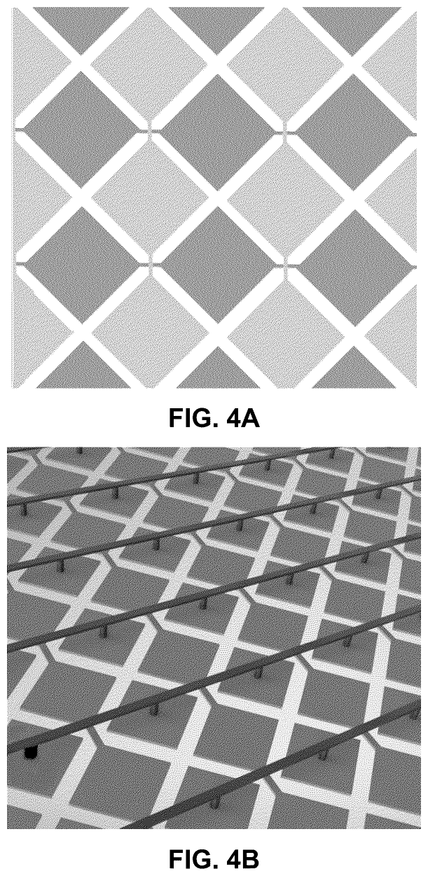

The cross-finger array is composed of row and column elements, each functioning as an individual channel. All squares reside on the primary metal layer, with each row element connecting to its corresponding square via a trace on the same layer. This cross-finger structure captures the two-dimensional coordinates of tongue movement with greater resolution than traditional single-electrode methods, as it allows for more channels within the same count. Traces on the second metal layer link the squares within each column element through dielectric via connections as shown in FIG. 4B. Additionally, every sensing element is linked to the ZIF (zero insertion force) connector using this second metal layer. By adopting this design, the total number of layers has been reduced from five to three compared to similar devices. This reduction in layer count decreases the overall thickness, thereby enhancing the device's flexibility and the wearer's comfort.

### Readout Circuit Design

A compact customized readout circuit has been designed and implemented to facilitate the integration of the SELMA device into the mouth. This circuit features wireless data transfer capabilities powered by a Bluetooth module, small enough to fit inside the mouse along with the SELMA device. The board connects to the SELMA sensor by ZIF connector, and the system is powered by a button lithium-ion battery. The sensing data, which is wirelessly transferred to the mobile app or PC, is visualized into a 3D column plot.

### Example 2: Sensor Testing

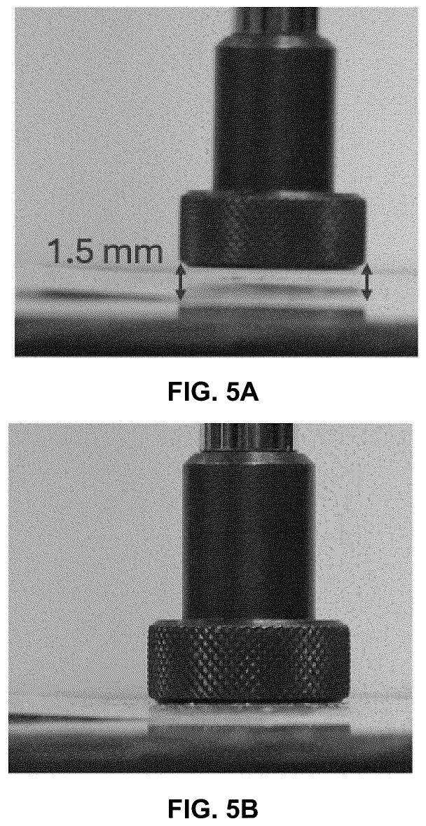

The sensor functions are separated into proximity and pressure sensing with different metrics. The proximity sensing measures relationship between distance and capacitance change while the pressure sensing focuses on pressure level and capacitance reading. Both experiments are conducted in the same condition where the pressure gauge and distance are measured. The experiment setup for the proximity sensing and pressure sensing is shown in FIGS. 5A-5B, respectively. The sensor pad is placed on a grounded flat copper plate while the conductive gauge head controls the distance and pressure. The conductive top and bottom setup is to mimic the property of tongue and upper palate. The sensor response is a converted capacitance reading that correlates with the circuit parameters. This converted reading is more sensitive than the raw capacitance reading, so the output of the sensor reading is directly used in the embodiments disclosed herein.

### Proximity Sensing

The proximity sensing range begins with slight contact between the gauge head and the sensor pad and extends to the point where the sensor reading decreases to zero, spanning from 0 to approximately 2.2 mm. As shown in FIG. 5A, the sensor reading in log scale is plotted with the distance change, the error is initially small and increases when it approaches the sensor detection limit.

### Pressure Sensing

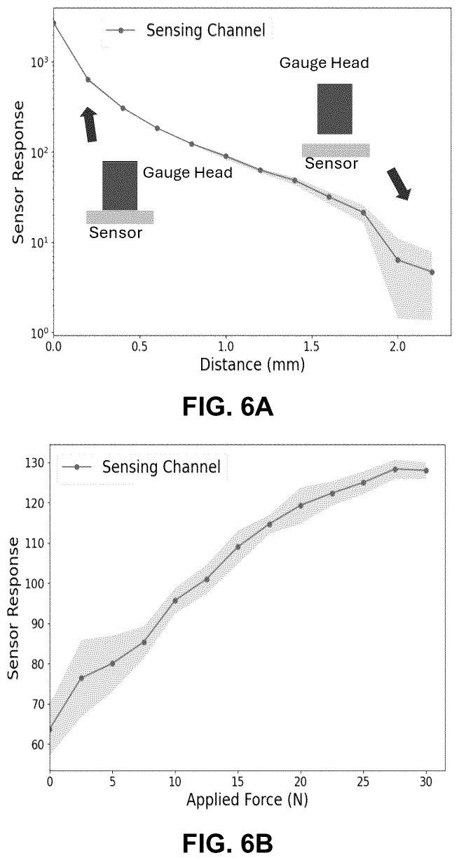

The pressure sensing capability of the SELMA system comes from both the increase in contact area due to material deformation (like the tongue) and the reduction in gap resulting from compression of the elastic layer on top of the sensor pad. While capacitance is not directly correlated with pressure, they nonetheless exhibit a linear relationship within the pressure range achievable by the tongue and palate. Since the distance between the gauge head and sensor pad is small and hence shows higher capacitance readings, raw capacitance values can be used for sensing. As shown in FIG. 6B, the linear relationship can be found between the force applied and the sensor reading.

Data Visualization with Mobile Interface

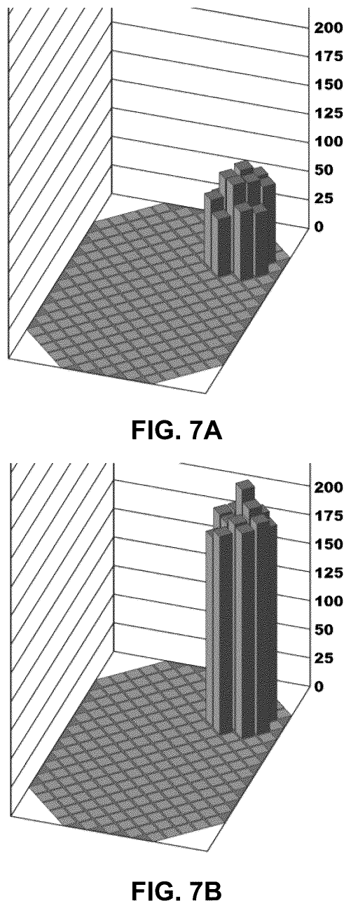

To better visualize the tongue position and movement, an interface to plot the sensor data into a real time 3D map has been developed. As shown in FIG. 7A, when the pressure gauge head approaches the sensor pad, the columns at the corresponding place start to rise and keep growing indicating the pressure gauge head enters the proximity sensing mode region. When the gauge head touches the sensor, the columns rise much faster as pressure applies which matches well with the single cell measurements.

### Example 3: Thin Film Device Fabrication

As the sensor array design and sensing function has been validated by the fPCB prototype, it is translated to a thinner and more flexible, biocompatible, and thermal plastic material platform such as using parylene. Parylene C can be comformally coated and is compatible with microfabrication. Also, it is widely used in medical electronic devices. Moreover, Parylene C can be thermally molded into intricate 3D shapes such as the contour of a pseudopalate, to achieve superior shape replication. This ensures improved contact between the device and the patient's palate. A test structure is designed with 3 layers of parylene, 2 metal layers and corresponding via and opening structure.

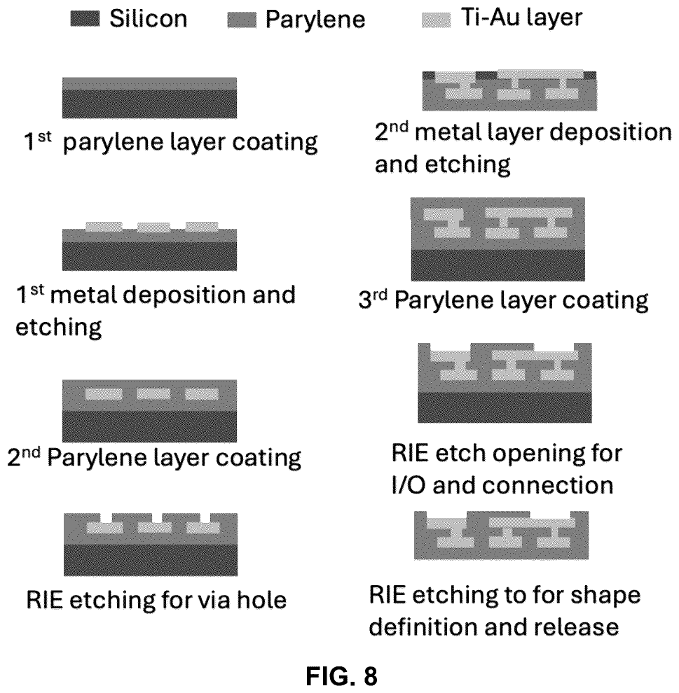

Microfabrication techniques using parylene are shown in FIG. 8. The fabrication process starts with a bare silicon wafer, which is used for a handle substrate (regardless of its orientation). The first 6 μm thick parylene layer is deposited on top of the wafer followed by a metal sputtering deposition with 50 nm of chromium and 200 nm gold. Then the pattern of the sensing array is transferred to the metal layer by wet etching. The X-axis of the sensor pad array is connected with metal lines on the first metal layer, while the vertical array and the connection between the sensor and I/O pin have to be achieved by the second layer metal trace and via holes. The second 6 μm thick parylene is deposited on top of the metal pad followed by a patterned RIE etching to open the via holes connecting the vertical array of the sensor pads. Then, the second metal layer with the same composition and thickness as those of the first metal layer is deposited using sputtering, followed by patterning using photolithography and wet etching to define the traces and vias for connection. The third layer of parylene is then coated on the device for passivation while additional photolithography and etching processes open the I/O pins.

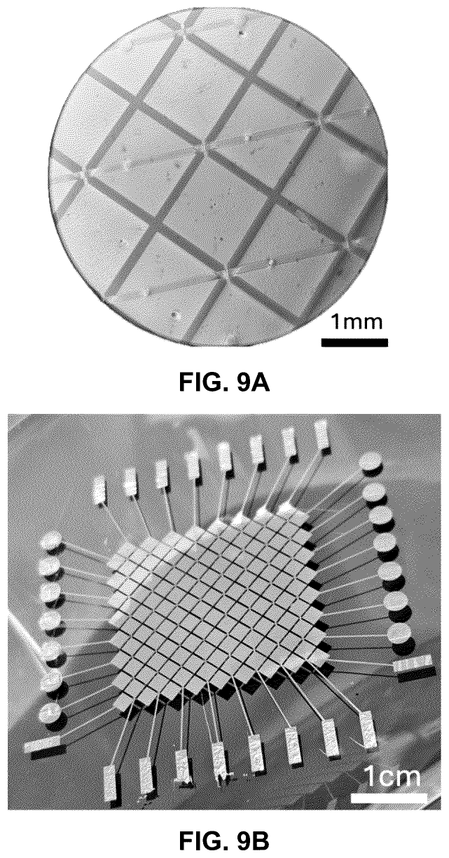

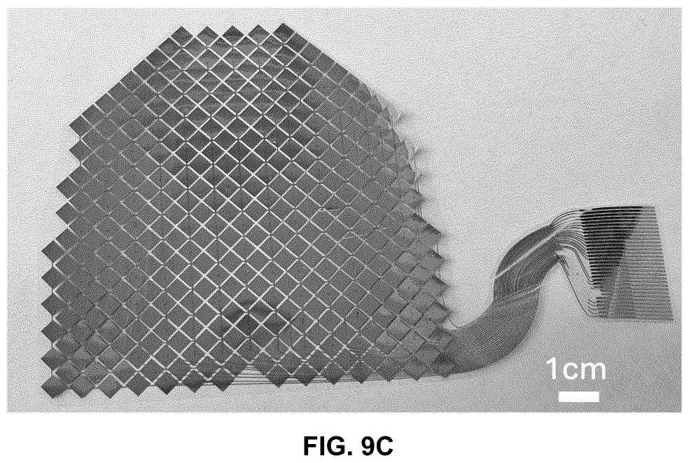

The final photolithography and RIE etching steps define the shape of the SELMA sensor and remove the surrounding parylene for the releasing purpose. Then the entire wafer is submerged into DI water and the parylene-metal sensor is clearly separated from the silicon due to the surface tension as shown in FIG. 9B and the details of sensor patterns in FIG. 9A.

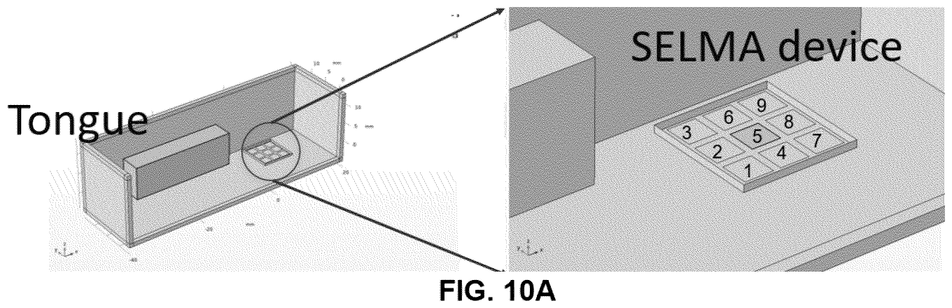

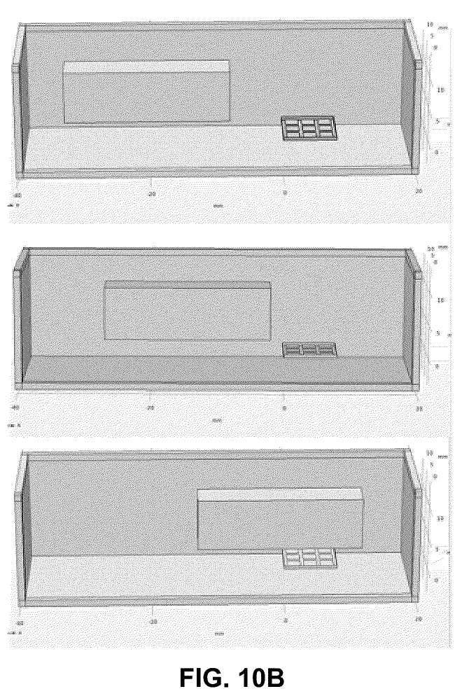

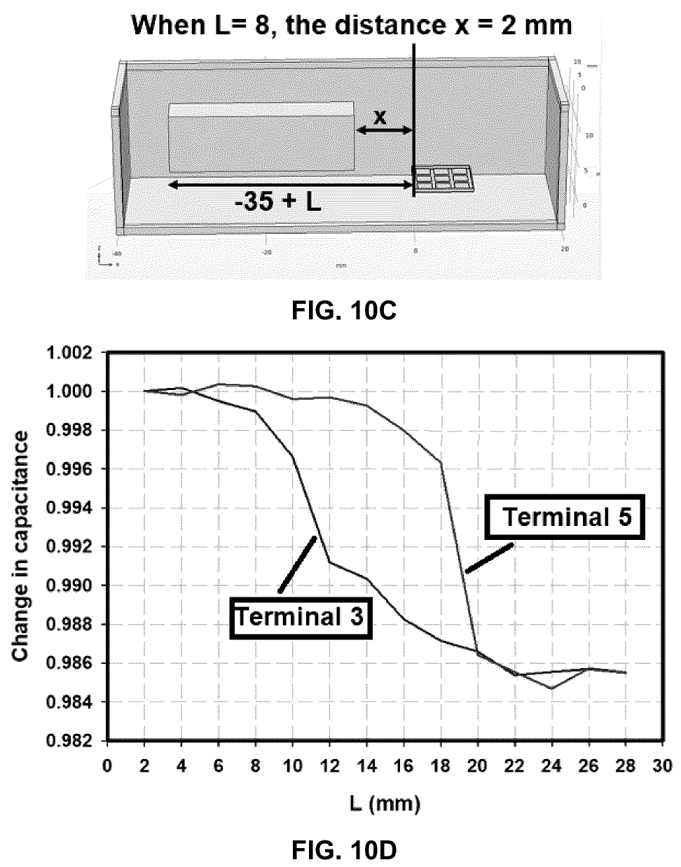

COMSOL simulations showing how the device can respond as the tongue approaches are shown in FIGS. 10A-10D.

The stand-alone parylene packaged sensor can now be molded into the upper palate shape for better wearing comfort and accurate sensing thanks to the thermal plastic property of parylene whose glass transition temperature is 80° C.

### Conclusion

In this research, a wearable, flexible, high resolution capacitive sensing system for tongue movement and position mapping powered by proximity, pressure and contact sensing modes has been developed. The design is first validated in a form of fPCB and then evolved to a microfabricated parylene platform. The SELMA EPG sensor system is one of the most versatile smart EPG to date thanks to its high channel counts, extremely thin thickness, and multi-modality sensing including proximity, contact, and pressure sensing modes.

With the validated process and approach, herein is disclosed a fully functional SELMA sensor system, featuring a palate-shaped parylene thin film, a higher channel count, and output pins compatible with the read-out board.

### Example 4: Board Design, Device Integration, and Device Performance

### Thin Film Device Integration

With the thin-film fabrication process now fully optimized, the new triple-mode capacitive sensor (proximity, contact, and pressure) can consistently be produced as an ultra-flexible membrane whose total thickness remains around 25 μm. which is an almost 50 times thickness reduction compared with commercial device and 10-fold thickness reduction compared with flexible printed circuit board solutions. The thin film enables better wearing comfort, improving both contact and detection accuracy.

Supporting Board Design and Integration with Thin Film Device

The PCB has been designed to have a low profile of 30 mm in length and 10 mm in width to fit comfortably in a patient mouth. The PCB integrates an Infineon Technologies PSOC™ 4 BL microcontroller capable of measuring the capacitance values of 30 individual channels. Signal acquisition is achieved through a 32-pin FPC connector interfaced with the sensor array. To enable RF communication, a 2.45 GHz chip antenna is matched via a pi network. Multiple decoupling capacitors are placed at the input power pints to stabilize the input voltage supplied by the lithium-ion battery. Additional onboard components, including crystal oscillators, are used internally by the microcontroller for clock regulation. A surface-mounted red LED provides a visual indication of active microcontroller operation.

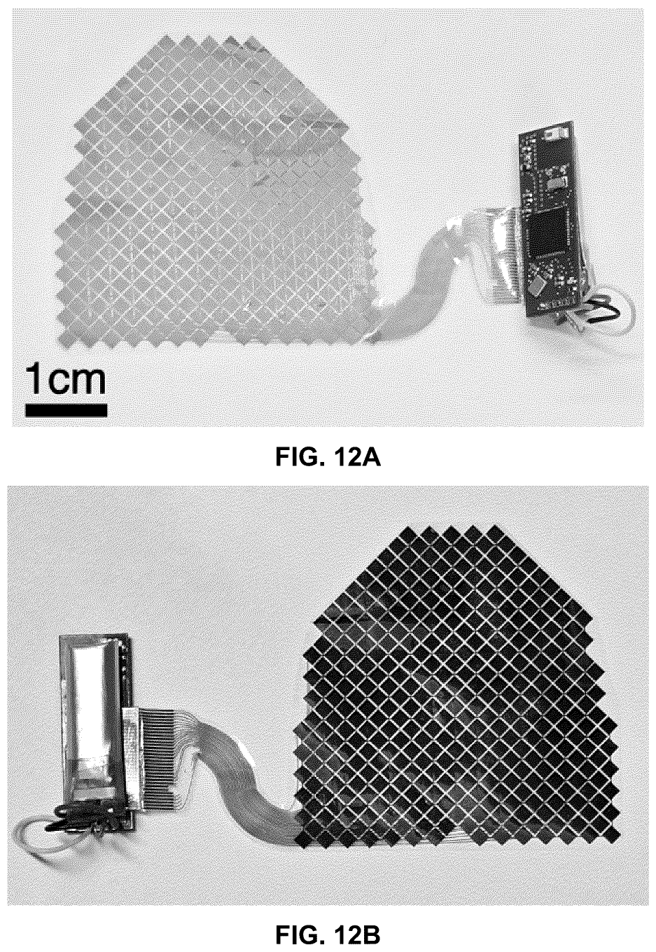

A lithium-ion battery is mechanically retained on the PCB with a low-profile cradle and spot-welded nickel tabs, as illustrated in FIGS. 12A-12B. After final assembly, the entire power block—including tabs, solder joints, and adjacent traces—is conformally encapsulated in a 2 μm parylene-C overcoat. This ultra-thin barrier prevents saliva from bridging exposed conductors while adding less than 0.1 g to the overall mass.

The power module is fully rechargeable. When the cell approaches end-of-charge, the parylene film is scored and lifted at the dedicated charge-pad window beside the positive lead, using a fine razor or scalpel. The exposed nickel pad mates with standard alligator or pogo-pin clips for trickle charging. After recharging, the area is resealed with another round of parylene coating, restoring the hermetic barrier without compromising device integrity or patient safety.

### Faster Read-Out System

The readout interface for acquiring data from the device has been optimized to achieve a response time as low as 7.6 ms. This enhancement enables reliable detection of both contact and pressure/proximity modes with reduced latency. These performance gains are realized through refined capacitance frequency measurement, implemented using the PSoC Creator IDE. The real time data from the microcontroller is transmitted wirelessly to a computer for visualization purposes where the data will be plotted.

### Sensor Capacitance Over Time

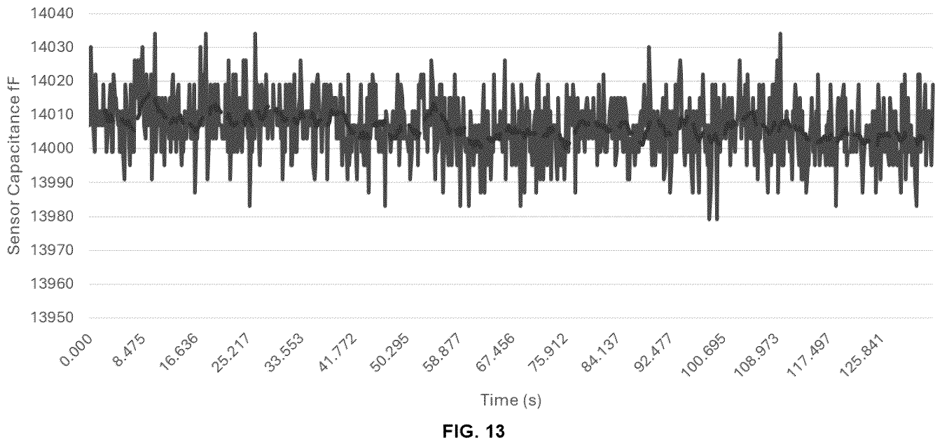

The sensor capacitance over time is shown in FIG. 13. The dataset consists of 800 data points collected over a span of 134 seconds. The instantaneous capacitance values are represented by the blue trace, while the red trace corresponds to a 25-point moving average for improved visualization of the overall trend. The sensor output exhibits high stability, with an average capacitance of 14,006.42 fF and a standard deviation of 9.32 fF. These results demonstrate the consistency and reliability of the capacitance measurements under 4N force steady-state conditions.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Andreasen Struijk, L. N. S. et al, “Wireless intraoral tongue control of an assistive robotic arm for individuals with tetraplegia,” J NeuroEngineering Rehabil, vol. 14, no. 1, p. 110, December 2017, doi: 10.1186/s12984-017-0330-2.
2. Baldoli, I., et al. (2017). A pressure-sensitive palatograph for speech analysis. Proceedings of the 39th annual international conference of the IEEE engineering in medicine and biology society (pp. 4431-4434).
3. Birkholz, P. et al, “Combined optical distance sensing and electropalatography to measure articulation,” in Interspeech 2011, ISCA, August 2011, pp. 285-288. doi: 10.21437/Interspeech.2011-111.
4. Chiu, W. S., et al. (1995). Quantitative measures of the palate using enhanced electropalatography. European Journal of Disorders of Communication, 30 (2), 149-160.
5. Dagenais, P. A. et al, Journal of Speech, Language, and Hearing Research, vol. 34, no. 6, pp. 1423-1435, 1991.
6. Fougeron, C., et al. (2000). 62 vs. 96 electrodes: A comparative analysis of reading and kay elemetrics EPG pseudo-palates. Seminar on speech production: Models and data CREST workshop on models on speech production: Motor planning and articulatory modelling (pp. 1-4).
7. Gibbon, F. E., et al. (2010). Visual feedback therapy with electropalatography. In A. L. Williams, S. McLeod, & R. J. McCauley (Eds.), Interventions for speech sound disorders in children (pp. 509-536). Paul H. Brookes Publishing Co.
8. Hardcastle, W. J., et al. (1991). Visual display of tongue-palate contact: Electropalatography in the assessment and remediation of speech disorders. British Journal of Disorders of Communication, 26 (1), 41-74.
9. Hori, K. et al., “Newly developed sensor sheet for measuring tongue pressure during swallowing,” Journal of prosthodontic research, vol. 53, no. 1, pp. 28-32, 2009.
10. Horne, R. et al, 2015. IET International Conference on Technologies for Active and Assisted Living (TechAAL), London, UK. doi: 10.1049/ic.2015.0141.
11. Horne, R. J., et al. (2015). Electropalatography as a human input device for patients suffering from spinal cord injuries. Proceedings of the IET international conference on Technologies for Active and Assisted Living (TechAAL) (pp. 1-4).
12. Huo, X., et al. (2008). A magneto-inductive sensor based wireless tongue-computer interface. IEEE Transactions on Neural Systems and Rehabilitation Engineering, 16 (5), 497-504. doi: 10.1109/TNSRE.2008.2003375
13. Jeon, S. et al, “Smart Electropalatography thin-film flexible pressure sensor array for Linguistic and Medical Applications,” Hilton Head Workshops, 2022.
14. Lee, A., et al. (2022). EPG activities in Japan and the impact of COVID-19 pandemic on EPG research and therapy: A report of presentations at the 7th EPG Symposium. International Journal of Language & Communication Disorders. Advance online publication.
15. Mat Zin, S. et al, Biomedical engineering online, vol. 20, pp. 1-19, 2021.
16. Matsumura, M., et al. (2002). Measurement of palatolingual contact pressure and tongue force using a force sensor mounted on a palatal plate. Biomechanisms, 16 (1), 75-85.
17. McAuliffe, M. J., et al. (2008). Influence of a standard electropalatography artificial palate upon articulation. Folia Phoniatrica Et Logopaedica, 60 (1), 45-53.
18. McLeod, S., et al. (2006). Adaptation to an electropalatograph palate: Acoustic, impressionistic, and perceptual data. American Journal of Speech-Language Pathology, 15 (2), 192-206.
19. Mirchandani, B., et al. (2020). Study of tongue-palate pressure patterns during the hold phase in the production of French dentialveolar and velar stops. Clinical Linguistics & Phonetics, 34 (1-2), 54-71.
20. Mohammad Azmi, H. H. et al, Acta oto-laryngologica, vol. 140, no. 10, pp. 838-844, 2020.
21. Murdoch, B. E., et al. (2004). Introducing the pressure-sensing palatograph—The next frontier in electropalatography. Clinical Linguistics & Phonetics, 18 (6-8), 433-445.
22. Pastore, G. “Tongue Position Tracking Device (TPTD): A Discreet Wireless Electropalatography and Glossometry Device.” University of Chicago Master's Thesis. 2018.
23. Sardini, E., et al. (2014). Analysis of tongue pressure sensor for biomedical applications. Proceedings of the 2014 IEEE international symposium on Medical Measurements and Applications (MeMeA) (pp. 1-5).
24. Saw, C. C. (1993). Customized 3-D electropalatography display. UCLA Working Papers in Phonetics, 85, 71-96.
25. Schöffmann, C. et al, IEEE Robotics and Automation Letters, vol. 7, no. 4, pp. 9929-9936, 2022.
26. Scobbie, J. M., et al. (2004). Advances in EPG for treatment and research: An illustrative case study. Clinical Linguistics & Phonetics, 18 (6-8), 373-389.
27. Sweeney, T., et al. (2016). Parent-led articulation therapy in cleft palate speech: A feasibility study. Journal of Clinical Speech and Language Studies, 23 (1), 21-41.
28. Tiede, M., et al. (2003). A new approach to pressure-sensitive palatography using a capacitive sensing device. In M. J. Sole, D. Recasens, & J. Romero (Eds.), Proceedings of the 15th International Congress of Phonetic Sciences (ICPhS) (pp. 3149-3152). Causal Productions.
29. Timmins, C. et al, Clinical linguistics & phonetics, vol. 25, no. 11-12, pp. 1022-1027, 2011.
30. Wong, M. N. et al, Journal of Medical Speech-Language Pathology, vol. 18, no. 3, pp. 24-34, 2010.

What is claimed is:

1. A pseudo-palate for capacitive sensing of tongue proximity to, tongue contact with, and tongue pressure against a hard palate of a subject, the pseudo-palate comprising an upper plurality of tracing lines and a lower plurality of tracing lines;
wherein the upper plurality of tracing lines and the lower plurality of tracing lines are embedded in a flexible material and separated by gap comprising the flexible material;
wherein the upper plurality of tracing lines and the lower plurality of tracing lines are connected by vias located at a plurality of intersection points, wherein the vias are filled with metal; and
wherein, when the pseudo-palate is worn by the subject, the upper plurality of tracing lines is closer to the hard palate than is the lower plurality of tracing lines.

2. The pseudo-palate of claim 1, wherein the capacitive sensing comprises fringing field capacitance sensing.

3. The pseudo-palate of claim 1, wherein the flexible material comprises parylene.

4. The pseudo-palate of claim 1, wherein the pseudo-palate has a thickness of less than about 30 μm.

5. The pseudo-palate of claim 1, wherein the upper plurality of tracing lines are perpendicular to the lower plurality of tracing lines.

6. The pseudo-palate of claim 1, wherein the pseudo-palate comprises at least 112 vias.

7. The pseudo-palate of claim 1, wherein the tracing lines comprise gold, titanium, platinum, nickel, chromium, copper, silver, or a combination thereof.

8. The pseudo-palate of claim 7, wherein the tracing lines comprise a titanium-gold (Ti—Au) alloy.

9. The pseudo-palate of claim 1, wherein the tracing lines comprise a width of from about 5 μm to about 50 μm.

10. The pseudo-palate of claim 1, further comprising a microprocessor, a wireless communication module, and a battery, wherein the microprocessor, wireless communication module, and battery are positioned on an outside wall of the subject's upper gums.

11. The pseudo palate of claim 1, further comprising a temperature sensor, an inertia sensor, an accelerometer, a gyroscope, a magnetometer, an infrared sensor for monitoring impacts, a heart rate monitor, or a combination thereof.

12. A method for diagnosing a neurological condition in a subject, the method comprising:
(a) fitting the subject with the pseudo-palate of claim 1;
(b) requiring the subject to perform at least one vocal task;
(c) collecting acoustic data and data from the pseudo-palate generated by the performance of the at least one vocal task;
(d) comparing the acoustic data and data from the pseudo-palate to a database of data from healthy subjects and subjects having diagnosed neurological conditions performing the same at least one vocal task; and
(e) diagnosing the neurological condition.

13. The method of claim 12, wherein the subject's tongue contacts the pseudo-palate and serves as an additional ground for the pseudo-palate, resulting in a higher electric field in a portion of the pseudo-palate contacted by the tongue.

14. The method of claim 12, wherein a decrease in distance between the subject's tongue and the pseudo-palate changes capacitance between the upper plurality of tracing lines and the lower plurality of tracing lines.

15. The method of claim 12, wherein tongue contact with the pseudo-palate compresses the flexible material, reducing the gap between the upper plurality of tracing lines and the lower plurality of tracing lines.

16. The method of claim 12, wherein the neurological condition comprises a traumatic brain injury, Parkinson's disease, dyspraxia, dysarthria, congenital sensory neuropathy, schizophrenia, depression, fatigue, stress, or a combination thereof.

17. The method of claim 16, wherein the traumatic brain injury comprises a concussion.

18. The method of claim 12, wherein the at least one vocal task comprises picture description, story-telling, or syllable repetition.

19. The method of claim 12, wherein the at least one vocal task further comprises an exercise task.

20. The method of claim 19, wherein the exercise task comprises walking on a treadmill or pedaling a stationary bicycle.
